SEQUENCE LISTING (6 sequences)

SEQ ID NO: 1 - protein, 28 aa, Artificial Sequence
  Mamilia Peptide
HSDAVFTDNYTRLRKQMAVKKYLNSILN

SEQ ID NO: 2 - protein, 170 aa, Artificial Sequence
  Mamilia Peptide
MDTRNKAQLLVLLTLLSVLFSQTSAWPLYRAPSALRLGDRIPFEGANEPDQVSLKEDIDMLQNALAENDTPYYDVSRNARHADGVFTSDFSKLLGQLSAKKYLESLMGKRVSSNISEDPVPVKRHSDAVFTDNYTRLRKQMAVKKYLNSILNGKRSSEGESPDFPEELEK

SEQ ID NO: 3 - protein, 169 aa, Artificial Sequence
  Mamilia Peptide
MDTRNKAQLLVLLTLLSVLFSQTSAWPLYRAPSALRLGDRIPFEGANEPDQVSLKEDIDMLQNALAENDTPYYDVSRNARHADGVFTSDFSKLLGQLSAKKYLESLMGKRVSNISEDPVPVKRHSDAVFTDNYTRLRKQMAVKKYLNSILNGKRSSEGESPDFPEELEK

SEQ ID NO: 4 - protein, 28 aa, Artificial Sequence
  Mamilia Peptide
  MOD_RES (1)..(1): ACETYLATION
  SITE (17)..(17): Oxidation of methionine
HSDAVFTDNYTRLRKQMAVKKYLNSILN

SEQ ID NO: 5 - protein, 28 aa, Artificial Sequence
  Mamilia Peptide
  MOD_RES (1)..(1): ACETYLATION
HSDAVFTDNYTRLRKQMAVKKYLNSVLN

SEQ ID NO: 6 - protein, 28 aa, Artificial Sequence
  Mamilia Peptide
  MOD_RES (1)..(1): ACETYLATION
  SITE (17)..(17): Oxidation of methionine
HSDAVFTDNYTRLRKQMAVKKYLNSVLN

(19) United States
(12) Patent Application Publication
(10) Pub. No.: US 2024/0181014 A1
(43) Pub. Date: Jun. 6, 2024

(54) USE OF AVIPTADIL ALONE OR IN COMBINATION WITH ALPHA LIPOIC ACID AS A THERAPEUTIC MEDICAMENT FOR POST-VIRAL INFECTION SYNDROME

(71) Applicant: ADVITA LIFESCIENCE AG, Genève (CH)

(72) Inventor: Dorian BEVEC, Genève (CH)

(21) Appl. No.: 18/556,073

(22) PCT Filed: Apr. 20, 2022
(86) PCT No.: PCT/IB2022/053709

§ 371 Date: Oct. 18, 2023

(30) Foreign Application Priority Data
Apr. 20, 2021 (EP) 21000109.5
Aug. 2, 2021 (EP) 21189203.9

(51) Int. Cl. A61K 38/22; A61K 31/381; A61K 35/12; A61K 48/00; A61P 31/14

(52) U.S. Cl. CPC A61K 38/2278; A61K 31/381; A61K 35/12; A61K 48/005; A61P 31/14

(57) ABSTRACT

The invention relates to the use of Aviptadil or a functional derivative or a precursor thereof alone or in combination with Alpha Lipoic Acid or a functional derivative or a precursor thereof as (a) therapeutic agent(s) for the prophylaxis and/or treatment of a post-viral infection syndrome including, without limitation, fibrotic disease, inflammatory disease, neurodegenerative disease, autoimmune disease, or heart and vascular disease as a consequence of post-viral infection syndrome, such as a SARS-COV-2 infection. The invention further relates to the use of the therapeutic agent(s) for intravenous administration, or for oral administration, or for administration by inhalation.

The invention relates to the use of the compound Aviptadil or a functional derivative or a precursor thereof alone or in combination with Alpha Lipoic Acid or a functional derivative or a precursor thereof as (a) therapeutic agent(s) for the prophylaxis and/or treatment of a post-viral infection syndrome including, without limitation, fibrotic disease, inflammatory disease, neurodegenerative disease, autoimmune disease, or heart and vascular disease as a consequence of post-viral infection syndrome, such as a SARS-CoV-2 infection.

The invention further relates to the use of the therapeutic agent(s) for intravenous administration, or for oral administration, or for administration by inhalation.

Post-viral infection syndrome is a disease that occurs after contracting a viral infection that can be life-altering. It can cause months, years or even a lifetime of debilitating symptoms that drastically reduce the quality of life. The following viruses are known to induce post-viral infection syndrome: Epstein-Barr virus, Human herpes virus 6, Human Immunodeficiency Virus, Influenza Virus, Enterovirus, Rubella, West Nile virus, Ross River virus, SARS, MERS, SARS-COV-2, Rhinovirus, Human Respiratory Syncytial Virus, Coxsackievirus.

Post-viral infection syndrome is a complex medical condition, characterized by long-term fatigue, sore throat, aches and pains across the body, concentration and memory problems, blood pressure changes, post-exertional malaise, gastric upsets such as irritable bowel syndrome, headaches, sleep disturbance, emotional instability, depression, dizziness, hypersensitivity to light, noise, temperature change, and touch, burning or prickling sensations in the limbs, and a prolonged loss of smell and taste.

These symptoms are to such a degree that they limit a person's ability to carry out ordinary daily activities. A key feature of the condition is that symptoms can suddenly worsen following only minimal physical or mental activity.

Post-viral infection syndrome is mainly caused by an increased levels of pro-inflammatory cytokines (which promote inflammation), nervous tissue inflammation and significant mitochondriopathies.

One example of the condition is the post-COVID-19 syndrome.

COVID-19 is caused by the coronavirus initially named “WH-Human-1 coronavirus”, “2019 novel coronavirus (2019-nCOV)”, or now widely accepted as “Coronavirus SARS-COV-2”, which emerged in Wuhan city and rapidly spread throughout the entire globe.

COVID-19 causes severe pulmonary injury to most or all of both lungs, leading to rapid onset of progressive malfunction of the lungs, especially with regard to the ability to take in oxygen, usually associated with the malfunction of other organs. This acute lung injury (ALI) condition is associated with extensive lung inflammation and accumulation of fluid in the alveoli that leads to low oxygen levels in the lungs. It is characterized by diffuse pulmonary microvascular injury resulting in increased permeability and, thus, non-cardiogenic pulmonary edema.

The clinical course of SARS-COV-2 infection appears to be extremely variable, from almost asymptomatic to severe pneumonia with multi-organ failure requiring critical care.

ALI is a predominant feature of acute SARS-COV-2 infection, and understanding the longer-term implications is critical given the large number of affected patients.

The most common radiological pattern of acute infection with SARS-COV-2 is of bilateral ground glass opacification with or without consolidation in a sub-pleural distribution, and a radiological and histological pattern of organising pneumonia pattern.

ALI is diagnosed based on signs and symptoms indicating progressively worsening respiratory functioning. The pathologic hallmark of the disease is diffuse alveolar damage, vascular endothelium damage, and damage to the surfactant-producing type II cells which results in loss of the integrity of the alveolar-capillary barrier, transudation of protein-rich fluid across the barrier, pulmonary edema, and hypoxemia from intrapulmonary shunting.

ALI is a medical emergency. Typically, patients require care in an intensive care unit (ICU). Symptoms usually develop within 24 to 48 hours of the original illness. The mortality rate is approximately 30-40%. Deaths usually result from multisystem organ failure rather than lung failure alone.

A vast variety of insults by the virus induce an inflammatory reaction of the pulmonary epithelium, endothelium, and invading neutrophils (PMN) together with a massive release of cytokines, chemokines, reactive oxygen species, lipid mediators, and growth factors. During the initial phase of the insult, a loss of endothelial and epithelial barrier function is found with concomitant invasion of neutrophils in the airspace. Loss of barrier function translates into an uncontrolled edema formation with proteins inhibiting the essential surfactant function translating in progressive hypoxia. While PMN are the first line of defense against invading microorganism, the release of their potent destructive means as proteases and reactive oxygen species may lead to ongoing damage to the host.

Since pulmonary fibrosis with persistent physiological deficit is a previously-described feature of patients recovering from similar coronaviruses, adequate treatment represents an early opportunity to modify the disease course, therefore potentially preventing irreversible impairment.

Following SARS-COV-2 pneumonitis, a cohort of patients are left with both radiological inflammatory lung disease and persistent physiological and functional deficit classified as interstitial lung disease, predominantly organising pneumonia.

It is an object of the present invention to provide therapeutic compounds effective for the prophylaxis and/or treatment of a post-viral infection syndrome such as post COVID-19 syndrome.

The causative mechanism of many diseases is the over activity of a biological pathway. A medication that can reduce the activity of the biological pathway can be effective in the prophylaxis and/or treatment of the disease caused by the over activity of the biological pathway. Similarly, the causative mechanism of many diseases is the over production of a biological molecule. A medication that can reduce the production of the biological molecule or block the activity of the over produced biological molecule can be effective in the prophylaxis and/or treatment of the disease caused by the over production of the biological molecule.

Conversely, the causative mechanism of many diseases is the under activity of a biological pathway. A medication that can increase the activity of the biological pathway can be effective in the prophylaxis and/or treatment of the disease caused by the under activity of the biological pathway. Also, similarly the causative mechanism of many diseases is the under production of a biological molecule. A medication that can increase the production of the biological molecule or mimic the biological activity of the under produced biological molecule can be effective in the prophylaxis and/or treatment of the disease caused by the under production of the biological molecule.

An object of the present invention is solved by the teaching of the independent claims. Further advantageous features, embodiments and details of the invention are evident from the dependent claims, the description, and the examples of the present application.

Accordingly, the invention relates to, inter alia, the following embodiments:
1. Aviptadil, a functional derivative or a precursor thereof, for use in the treatment of a post-viral infection syndrome.
2. A vector encoding Aviptadil, a functional derivative or a precursor thereof, for use of embodiment 1, for use in the treatment of a post-viral infection syndrome.
3. A biological cell comprising the vector for use of embodiment 2, for use in the treatment of a post-viral infection syndrome.
4 A composition comprising Aviptadil, functional derivative or precursor for use of embodiment 1, the vector for use of embodiment 2 or the biological cell for use of embodiment 3, and alpha-lipoic acid, a functional derivative or a precursor thereof, for use in the treatment of a post-viral infection syndrome.
5 A pharmaceutical product comprising Aviptadil, functional derivative or precursor for use of embodiment 1, the vector for use of embodiment 2 or the biological cell for use of embodiment 3, or the composition for use of embodiment 4, and a pharmaceutically acceptable carrier, excipient and/or diluents for use in the treatment of a post-viral infection syndrome.
6. Aviptadil, functional derivative or precursor for use of embodiment 1, the vector for use of embodiment 2, the biological cell for use of embodiment 3, the composition for use of embodiment 4 or the pharmaceutical product for use of embodiment 5, wherein the treatment of post-viral infection syndrome is the treatment and/or prevention of at least one disease or disorder selected from the group consisting of an autoimmune disease, a fibrotic disease, an inflammatory disease, a neurodegenerative disease, an infectious disease, a lung disease and a heart and vascular disease.
7. Aviptadil, functional derivative or precursor for use of embodiment 1 or 6, the vector for use of embodiment 2 or 6, the biological cell for use of embodiment 3, the composition for use of embodiment 4 or 6 or the pharmaceutical product for use of embodiment 5 or 6, wherein the post-viral infection syndrome is post-COVID-19 syndrome.
9. Aviptadil, functional derivative or precursor for use of embodiment 7, the vector for use of embodiment 7, the biological cell for use of embodiment 7, the composition for use of embodiment 7 or the pharmaceutical product for use of embodiment 7, for use in treatment of at least one symptom of post-COVID-19 syndrome selected from the group consisting of fatigue, somnolence, headaches, dizziness, cerebrovascular disease, seizures, neuropathy and encelopathy.
10. Aviptadil, functional derivative or precursor for use of any of the previous embodiments, the vector for use of any of the previous embodiments, the biological cell for use of any of the previous embodiments, the composition for use of any of the previous embodiments or the pharmaceutical product for of use any of the previous embodiments, for use in oral, intravenous, inhalation and/or intranasal treatment.
11. Aviptadil, functional derivative or precursor for use of embodiment 10, the vector for use of embodiment 10, the biological cell for use of embodiment 10, the composition for use of embodiment 10 or the pharmaceutical product for use of embodiment 10, for use in intranasal treatment.
12. Aviptadil, functional derivative or precursor for use of embodiment 11, the vector for use of embodiment 11, the biological cell for use of embodiment 11, the composition for use of embodiment 11 or the pharmaceutical product for use of embodiment 11, wherein the intranasal administration is direct nose-to-brain administration.

Accordingly, in one embodiment, the invention relates to Aviptadil, or a functional derivative or a precursor thereof, for use in the treatment of a post-viral infection syndrome.

The term Aviptadil” or “VIP” (vasoactive intestinal peptide), as used herein, refers to a peptide comprising the sequence defined by the SEQ ID NO: 1.

The term “functional derivative”, as used herein, refers to any molecule that can be derived from a parent molecule, wherein both the parent molecule and the derivative have the ability to bind at least one receptor of interest. The biological effects of Aviptadil are mediated by G protein-coupled receptors, VPAC1, VPAC2 and the PAC1 receptor. In some embodiments, the functional derivative described herein is a functional derivative of VIP in that it binds to the receptor(s) VPAC1, VPAC2, and/or PAC1, preferably to VPAC1, VPAC2, and PAC1. In some embodiments the parent molecule described herein is VIP or a peptide comprising a sequence as defined by the SEQ ID NO: 2 or SEQ ID NO: 3. In some embodiments the functional derivative described herein consists of or comprises a sequence as defined by the SEQ ID NO: 4, SEQ ID NO: 5, SEQ ID NO: 6. For example, functional derivatives of Aviptadil can be prepared by derivatizing with one or more functional group, by amino acid deletions, insertion of 1, 2, 3, 4, 5, 6, or 7 amino acids and/or by replacing 1, 2, 3, 4, 5, 6 or 7 amino acids and/or by any other method known to the person skilled in the art (see e.g. in U.S. Pat. No. 8,329,640, EP3311828; Campos-Salinas, J., et al., 2014, The Journal of biological chemistry, 289(21), 14583-14599). In some embodiments, the derivative compared to the parent molecule has at least one property selected from the group consisting of increased stability, increased half-life, altered ionic charge, altered hydrophobicity index, altered percentage of α-helix, increased specificity.

The term “precursor”, as used herein, refers to any molecule(s) that can be turned into an active component by a chemical reaction. In some embodiments, the chemical reaction turning the precursor into an active component occurs before or during the administration process (e.g. in an administration device or in the aerosol). In some embodiments, the precursor described herein is a prodrug and the chemical reaction occurs in the body. In some embodiments, the chemical reaction turning the precursor into an active component is catalyzed by an enzyme of the body, preferably an enzyme expressed by cells of the respiratory tract and/or the central nervous system. In some embodiments, the precursor described herein is pre-pro Aviptadil. The production of precursors of Aviptadil is known to the person skilled in the art (see, e.g. Simoncsits A, et al., 1988, Eur J Biochem. December 15; 178(2):343-50.)

The term “post-viral infection syndrome”, as used herein, refers to a disease or disorder or a symptom thereof, which occurs after contracting a viral infection. The post-viral infection syndrome can comprise new and/or persistent symptoms compared to the acute viral infection. In some embodiments, the post-viral infection syndrome described herein occurs at least 7, 8, 9, 10, 11, 12, 13, 14, 15, 16, 17, 18, 19, 20, 21, 22, 23, 24, 25, 26, 27 or 28 days, 5, 6, 7, 8, 10, 11, 12, 13, 14, 15, 16 weeks after contracting a viral infection. In some embodiments, the post-viral infection syndrome described herein occurs in a subject after the subject contracting a viral infection is contagious. In some embodiments, the post-viral infection syndrome described herein is a disease or disorder or a symptom thereof, which occurs in a virus-negative disease or disorder, wherein the virus is not detectable anymore after an infection. Methods for detecting viruses are known to the person skilled in the art. In some embodiments, the virus detectability described herein is the detectability by a method selected from the group of virus isolation, nucleic acid-based methods, microscopy-based methods, host antibody detection, electron microscopy and host cell phenotype. In some embodiments, the virus detectability described herein is the detectability by a PCR-based method.

### Infectious Disease

The immune system in higher vertebrates represents the first line of defense against various antigens that can enter the vertebrate body, including microorganisms such as bacteria, fungi and viruses that are the causative agents of a variety of diseases.

Despite large immunization programs, viral infections, such as influenza virus, Coronavirus SARS-COV-2, human immunodeficiency virus (“HIV”), herpes simplex virus (“HSV”, type 1 or 2), human papilloma virus (“HPV”, type 16 or 18), human cytomegalovirus (“HCMV”) or human hepatitis B or C virus (“HBV”, Type B; “HCV”, type C) infections, will remain a serious source of morbidity and mortality throughout the world and a significant cause of illness and death among people with immune-deficiency associated with aging or different clinical conditions. The ability of viruses to rapidly mutate the target proteins represents an obstacle for effective treatment with molecules which selectively inhibit the function of specific viral polypeptides. Thus, there is need for new therapeutic strategies to prevent and treat viral infections and the long-term consequences of such infections.

In some embodiments, the viral infection described herein is an infection of a virus selected from the group consisting of a DNA virus (double or single stranded), an RNA virus (single or double stranded, whether positive or negative), a reverse transcribing virus and any emerging virus (enveloped or non-enveloped).

In some embodiments, the viral infection described herein is an infection selected from the group consisting of Adenovirus, Rhinovirus, RSV, Influenza virus, Parainfluenza virus, Metapneumovirus, Coronavirus, Enterovirus, Adenovirus, Bocavirus, Polyomavirus, Herpes simplex virus, Cytomegalovirus, Bocavirus, Polyomavirus, and Cytomegalovirus.

The RNA virus described herein may be an enveloped or coated virus or a nonenveloped or naked RNA virus. The RNA virus may be single stranded RNA (ssRNA) virus or a double stranded RNA (dsRNA) virus. The single stranded RNA virus may be a positive sense ssRNA virus or a negative sense ssRNA virus.

In some embodiments, the RNA virus described herein is selected from the group consisting of Rhinovirus, RSV, Influenza virus, Parainfluenza virus, Metapneumovirus, Coronavirus, Enterovirus Adenovirus, Bocavirus, Polyomavirus, Herpes simplex virus, and Cytomegalovirus.

In some embodiments, the Coronavirus described herein is a Coronavirus from the genus selected from the group consisting of α-CoV, β-CoV, γ-CoV or δ-CoV. In some embodiments, the Coronavirus described herein is of the genus α-CoV or β-CoV. In some aspects, the Coronavirus described herein is selected from the group consisting of Human coronavirus OC43 (HCoV-OC43), Human coronavirus HKU1 (HCoV-HKU1), Human coronavirus 229E (HCoV-229E), Human coronavirus NL63 (HCoV-NL63, New Haven coronavirus), Middle East respiratory syndrome-related coronavirus (MERS-COV or “novel coronavirus 2012”), Severe acute respiratory syndrome coronavirus (SARS-COV or “SARS-classic”), and Severe acute respiratory syndrome coronavirus 2 (SARS-COV-2 or “novel coronavirus 2019”).

In some embodiments, the viral infection described herein is an infection of a virus selected from the group consisting of Epstein-Barr virus, human herpes virus 6, Human Immunodeficiency Virus, Influenza Virus, Enterovirus, Rubella, West Nile virus, Ross River virus, SARS, MERS, SARS-COV-2, Rhinovirus, Human Respiratory Syncytial Virus and Coxsackievirus.

The invention provides the means and method to treat a post-viral infection syndrome using VIP (Examples 1 to 4). VIP binds primarily to the VPAC1 and VPAC2 receptors (Laburthe, M., et al., 2007, Peptides, 28(9), 1631-1639). Therefore, the binding of VIP can reverse virus-induced damage and inhibit long-term symptoms such as inflammatory processes associated with the viral infection.

Accordingly, the invention is at least in part based on the surprising finding that the binding of VIP can have a disease-modifying impact on post-viral infection syndrome.

In certain embodiments, the invention relates to a vector encoding Aviptadil, a functional derivative or a precursor thereof, for use of the invention, for use in the treatment of a post-viral infection syndrome.

The term “vector”, as used herein, refers to a nucleic acid molecule, capable of transferring or transporting another nucleic acid molecule. The transferred nucleic acid is generally linked to, i.e., inserted into, the vector nucleic acid molecule. A vector may include sequences that direct autonomous replication in a cell, or may include sequences sufficient to allow integration into a cell DNA. Useful vectors include, for example, plasmids (e.g., DNA plasmids or RNA plasmids), transposons, cosmids, bacterial artificial chromosomes, and viral vectors.

In some embodiments, the vector described herein is transfected with the support of a transfection enhancer, e.g., a transfection enhancer selected from the group consisting of oligonucleotides, lipoplexes, polymersomes, polyplexes, dendrimers, inorganic nanoparticles and cell-penetrating peptides.

The production of vectors encoding VIP is known to the person skilled in the art (see, e.g. Simoncsits A, et al., 1988, Eur J Biochem. December 15; 178(2):343-50.). In certain embodiments, the vector for use of the invention is a vector for transient transfection.

In certain embodiments, the vector for use of the invention is a vector for stable transfection.

In some embodiments, the invention relates to the vector for use of the invention, wherein the vector is administered in doses in the range from at least 10⁴, 10⁵, 10⁶, 10⁷, 10⁸, 10⁹, 10¹⁰, 10¹¹, 10¹², 10¹³, 10¹⁴, 10¹⁵, 10¹⁶, or more vector genomes per kilogram (vg/kg) of the weight of the subject, to achieve a therapeutic effect.

In some embodiments, the vector for use of the invention induces local expression of VIP, e.g. local expression of VIP in cells of the lung.

Therefore, by expressing VIP in cells of the subject to be treated a prolonged and/or local effect is achieved, whereby the disease-modifying effect (Examples 1-4) can be achieved particularly well.

In certain embodiments, the invention relates to a biological cell comprising the vector for use of the invention, for use in the treatment of a post-viral infection syndrome.

The term “biological cell”, as used herein, refers to a cell into which exogenous nucleic acid has been introduced, including the progeny of such cells.

In certain embodiments, the invention relates to the biological cell for use of the invention, wherein a clinically relevant number or population of biological cells, e.g., at least 10⁴, 10⁵, 10⁶, 10⁷, 10⁸, 10⁹, typically more than 10⁹ or at least 10¹⁰ cells per dose are administered. The number of cells will depend upon the use for which the composition, the pharmaceutical product or the biological cell of the invention is intended as will the type of cell. For uses provided herein, the cells are typically in a volume of a liter or less, can be 500 ml or less, even 250 ml or 100 ml or less. Hence the density of the desired cells is typically greater than 10⁶ cells/ml and generally is greater than 10⁷ cells/ml. The clinically relevant number of biological cells can be apportioned into multiple infusions that cumulatively equal or exceed 10⁹, 10¹⁰ or 10¹¹ cells. The total dose of the biological cell of the invention for one therapy cycle is typically about 1×10+ cells/kg to 1×10¹⁰ cells/kg biological cells or more, depending on the factors for consideration mentioned above.

Biological cells can be used to induce local treatment of VIP by cell migration (e.g. migration through the blood-brain barrier) and/or cell adhesion (e.g. adhesion to lung tissue).

Therefore, by expressing VIP in biological cells a local and prolonged expression can be achieved, whereby the disease-modifying effect (examples 1-4) can be achieved particularly well.

In certain embodiments, the invention relates to a composition comprising the Aviptadil, functional derivative or precursor for use of the invention, and alpha-lipoic acid, a functional derivative or a precursor thereof, for use in the treatment of a post-viral infection syndrome.

In certain embodiments, the invention relates to the vector for use of the invention, and alpha-lipoic acid, a functional derivative or a precursor thereof, for use in the treatment of a post-viral infection syndrome.

In certain embodiments, the invention relates to the biological cell for use of the invention, and alpha-lipoic acid, a functional derivative or a precursor thereof, for use in the treatment of a post-viral infection syndrome.

In some embodiments, the alpha lipoic acid described herein refers to (R)-(+) -alpha lipoic acid, (S)-(−)-alpha lipoic acid or to any mixture of (R/S)-alpha lipoic acid such as a racemic mixture of (R/S)-lipoic acid.

Functional derivatives and precursors of alpha lipoic acid are known to the person skilled in the art (see e.g. US20110212954).

In certain embodiments, the Aviptadil, analog or derivative for use of the invention, the vector for use of the invention or the biological cell for use of the invention, and alpha-lipoic acid, are used for the treatment of a post-viral infection syndrome in a combination administration.

The invention provides the means and method to treat a post-viral infection syndrome using VIP in combination with alpha lipoic acid (Examples 1 to 4). Therefore, the combination of VIP and alpha lipoic acid can reverse virus-induced damage and inhibit long-term symptoms such as inflammatory processes associated with the viral infection.

Accordingly, the invention is at least in part based on the surprising finding that the binding of VIP and alpha lipoic acid can have a disease-modifying impact on post-viral infection syndrome.

In certain embodiments, the invention relates to a pharmaceutical product comprising the Aviptadil, functional derivative or precursor for use of the invention and a pharmaceutically acceptable carrier, excipient and/or diluents for use in the treatment of a post-viral infection syndrome.

In certain embodiments, the invention relates to a pharmaceutical product comprising the vector for use of the invention and a pharmaceutically acceptable carrier, excipient and/or diluents for use in the treatment of a post-viral infection syndrome.

In certain embodiments, the invention relates to a pharmaceutical product comprising the biological cell for use of the invention and a pharmaceutically acceptable carrier, excipient and/or diluents for use in the treatment of a post-viral infection syndrome.

In certain embodiments, the invention relates to a pharmaceutical product comprising the composition for use of the invention, and a pharmaceutically acceptable carrier, excipient and/or diluents for use in the treatment of a post-viral infection syndrome.

A “pharmaceutically acceptable carrier, excipient and/or diluent”, as used herein, refers to an ingredient in the pharmaceutical product, other than the active ingredient(s), which is nontoxic to recipients at the dosages and concentrations employed.

In some embodiments the pharmaceutically acceptable carrier is at least one selected from the group consisting of buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as polyethylene glycol (PEG).

In certain embodiments, the invention relates to the Aviptadil, functional derivative or precursor for use of the invention, wherein the treatment of post-viral infection syndrome is the treatment and/or prevention of at least one disease or disorder selected from the group consisting of an autoimmune disease, a fibrotic disease, an inflammatory disease, a neurodegenerative disease, an infectious disease, a lung disease, and a heart and vascular disease.

In certain embodiments, the invention relates to the vector for use of the invention, wherein the treatment of post-viral infection syndrome is the treatment and/or prevention of at least one disease or disorder selected from the group consisting of an autoimmune disease, a fibrotic disease, an inflammatory disease, a neurodegenerative disease, an infectious disease, a lung disease, and a heart and vascular disease.

In certain embodiments, the invention relates to the biological cell for use of the invention, wherein the treatment of post-viral infection syndrome is the treatment and/or prevention of at least one disease or disorder selected from the group consisting of an autoimmune disease, a fibrotic disease, an inflammatory disease, a neurodegenerative disease, an infectious disease, a lung disease, and a heart and vascular disease.

In certain embodiments, the invention relates to the composition for use of the invention, wherein the treatment of post-viral infection syndrome is the treatment and/or prevention of at least one disease or disorder selected from the group consisting of an autoimmune disease, a fibrotic disease, an inflammatory disease, a neurodegenerative disease, an infectious disease, a lung disease, and a heart and vascular disease.

In certain embodiments, the invention relates to the pharmaceutical product for use of the invention, wherein the treatment of post-viral infection syndrome is the treatment and/or prevention of at least one disease or disorder selected from the group consisting of an autoimmune disease, a fibrotic disease, an inflammatory disease, a neurodegenerative disease, an infectious disease, a lung disease, and a heart and vascular disease.

### Autoimmune Disease

Autoimmune disease refers to any of a group of diseases or disorders in which tissue injury is associated with a humoral and/or cell-mediated immune response to body constituents or, in a broader sense, an immune response to self. The pathological immune response may be systemic or organ-specific. That is, for example, the immune response directed to self may affect joints, skin, myelin sheath that protects neurons, kidney, liver, pancreas, thyroid, adrenals, and ovaries.

It has long been known that immune complex formation plays a role in the etiology and progression of autoimmune disease. For example, inflammation in patients with arthritis has long been considered to involve phagocytosis by leukocytes of complexes of antigen, antibody and complement-immune complexes. However, now it is recognized that inflammation caused by immune complexes in the joints (arthritis), the kidneys (glomerulonephritis), and blood vessels (vasculitis) is a major cause of morbidity in autoimmune diseases. Increased immune complex formation correlates with the presence of antibodies directed to self or so-called autoantibodies, and the presence of the latter can also contribute to tissue inflammation either as part of an immune complex or unbound to antigen (free antibody). In some autoimmune diseases, the presence of free autoantibody contributes significantly to disease pathology. This has been clearly demonstrated for example in SLE (anti-DNA antibodies), immune thrombocytopenia (antibody response directed to platelets), and to a lesser extent rheumatoid arthritis (IgG reactive rheumatoid factor). The important role of immune complexes and free autoantibodies is further demonstrated by the fact that successful treatment of certain autoimmune diseases has been achieved by the removal of immune complexes and free antibody by means of specific immunoadsorption procedures.

Another aspect of the etiology and progression of autoimmune disease is the role of pro-inflammatory cytokines. Under normal circumstances, pro-inflammatory cytokines such as tumor necrosis factor (TNF) and interleukin-1 (IL-1) play a protective role in the response to infection and cellular stress. However, the pathological consequences which result from chronic and/or excessive production of TNF and IL-1 are believed to underlie the progression of many autoimmune diseases such as rheumatoid arthritis, Crohn's disease, inflammatory bowel disease, and psoriasis. Other pro-inflammatory cytokines include interleukin-6, interleukin-8, interleukin-17, and granulocyte-macrophage colony-stimulating factor.

Naturally occurring CD4+CD25+ regulatory T cells (Tregs) play a critical role in the control of periphery tolerance to self-antigens. Interestingly, they also control immune responses to allergens and transplant antigens. Studies in animal models have shown that adoptive transfer of CD4+CD25+ Tregs can prevent or even cure allergic and autoimmune diseases, and appear to induce transplantation tolerance. Thus, adoptive cell therapy using patient-specific CD4+CD25+ Tregs or medications that induce or regulate those cells has emerged as an individualized medicine for the treatment of inflammatory disease including allergy, autoimmune disease and transplant rejection.

The interaction of leukocytes with the vessel endothelium to facilitate the extravasation into the tissue represents a key process of the body's defense mechanisms. Excessive recruitment of leukocytes into the inflamed tissue in chronic diseases like autoimmune disorders could be prevented by interfering with the mechanisms of leukocyte extravasation. Significant progress in elucidating the molecular basis of the trafficking of leukocytes from the blood stream to the extravascular tissue has been achieved that enables new strategies for therapeutic approaches. The multistep process of leukocyte rolling, firm adhesion and transmigration through the endothelial wall is facilitated by a dynamic interplay of adhesion receptors on both leukocytes and on endothelial cells as well as chemokines.

Examples of autoimmune diseases of the eyes are idiopathic opticus-neuritis, ophthalmia sympathica, anterior uveitis and other uveitis forms, retina degeneration, and Mooren's ulcer.

Examples of autoimmune diseases of the skin are bullous pemphigoides, chronic urticaria (autoimmune subtype), dermatitis herpetiformis (morbus Duhring), epidermolysis bullosa aquisita (EBA), acquired angioedema, herpes gestationes, hypocomplementemic urticarial vasculitis syndrome (HUVS), linear IgA-dermatosis, and pemphigus.

Examples of hematological autoimmune diseases are autoimmune hemolytic anemia, autoimmune neutropenia, Evans syndrome, inhibitor hemophilia, idiopathic thrombocytopenial purpura (ITP) and pernicious anemia.

Examples of autoimmune diseases of the heart are congenital heart block, idiopathic dilatative cardiomyopathy, peripartum-cardiomyopathy, postcardiotomy syndrome, and postinfarct syndrome (Dressler syndrome).

Examples of autoimmune diseases of the ear, nose and throat are chronic sensorineural hearing loss and morbus Menière.

Examples of autoimmune diseases of the colon are autoimmune enteropathy, colitis ulcerosa, indeterminant colitis, Crohn's disease and gluten-sensitive enteropathy.

Examples of neurological autoimmune disorders are Guillain-Barré syndrome, IgM gammopathy-associated neuropathy, Lambert-Eaton syndrome, Miller-Fisher syndrome, multiple sclerosis, multifocal motoric neuropathy, myasthenia gravis, paraneoplastic neurological syndrome, Rasmussen's encephalitis, and stiff-man syndrome.

Examples of autoimmune diseases of the kidney are anti-TBM-nephritis, Goodpasture's syndrome/anti-GBM-nephritis, IgA-nephropathy, interstitial nephritis, and membrane proliferative glomerulonephritis.

Further diseases that may be caused by an autoimmune reaction are Behcet disease, chronic fatigue immune dysfunction syndrome (CFIDS), Cogan syndrome I, endometriosis, HELLP syndrome, Bechterew's disease, polymyalgia rheumatica, psoriasis, sarcoidosis and vitiligo.

### Fibrotic Disease

Fibrosis or fibrosis associated disorder affects the liver, epidermis, endodermis, muscle, tendon, cartilage, heart, pancreas, lung, uterus, nervous system, testis, ovary, adrenal gland, artery, vein, colon, small intestine, biliary tract, or stomach. In a further embodiment, the fibrosis or fibrosis associated disorder is interstitial lung fibrosis. In another embodiment, the fibrosis or fibrosis associated disorder is the result of an infection with Coronavirus SARS-COV-2. In another embodiment, the fibrosis or fibrosis associated disorder is the result of wound healing e.g., after acute lung injury.

Fibrosis is generally characterized by the pathologic or excessive accumulation of collagenous connective tissue.

Diseases associated with fibrosis include acute lung injury and acute respiratory distress syndrome (including bacterial pneumonia induced, viral pneumonia induced, ventilator induced, non-pulmonary sepsis induced, and aspiration induced).

### Increased Number of Activated Myofibroblasts in Fibrotic Diseases

The emergence and disappearance of the myofibroblast appears to correlate with the initiation of active fibrosis and its resolution, respectively. In addition, the myofibroblast has many phenotypic features, which embody much of the pathologic alterations in fibrotic tissue, e.g., lung tissue. These features would seem to argue for an important role for the myofibroblast in the pathogenesis of fibrosis, e.g., lung fibrosis. Furthermore, the persistence of the myofibroblast may herald progressive disease, and, conversely, its disappearance may be an indicator of resolution. This in turn suggests that future therapeutic strategies targeting the myofibroblast would be productive.

Patients usually exhibit evidence of active fibrosis with increased numbers of activated fibroblasts, many of which have the phenotypic characteristics of myofibroblasts. At these sites, increased amounts of extracellular matrix deposition are evident with effacement of the normal alveolar architecture. Animal model studies show the myofibroblast to be the primary source of type I collagen gene expression in active fibrotic sites. In vitro studies show differentiation of these cells from fibroblasts under the influence of certain cytokines but indicate their susceptibility to nitric oxide mediated apoptosis. In addition to promoting myofibroblast differentiation, transforming growth factor-ß1 (TGF-ß1) provides protection against apoptosis. Thus, this well-known fibrogenic cytokine is important both for the emergence of the myofibroblast and its survival against apoptotic stimuli. This is consistent with the critical importance of this cytokine in diverse models of fibrosis in various tissues. In view of these properties, the persistence or prolonged survival of the myofibroblast may be the key to understanding why certain forms of lung injury may result in progressive disease, terminating in end stage disease.

Although pulmonary fibrosis has diverse etiologies, there is a common feature characteristic of this process, namely, the abnormal deposition of extracellular matrix that effaces the normal lung tissue architecture. A key cellular source of this matrix is the mesenchymal cell population that occupies much of the fibrotic lesion during the active period of fibrosis. This population is heterogeneous with respect to a number of key phenotypes. One of these phenotypes is the myofibroblast, which is commonly identified by its expression in α-smooth muscle actin and by features that are intermediate between the bona fide smooth muscle cell and the fibroblast. The de novo appearance of myofibroblasts at sites of wound healing and tissue repair/fibrosis is associated with the period of active fibrosis and is considered to be involved in wound contraction. Furthermore, the localization of myofibroblasts at sites undergoing active extracellular matrix deposition suggests an important role for these cells in the genesis of the fibrotic lesion.

### Increased TGF-β₁ Family Levels in Fibrotic Diseases

The transforming growth factor-β₁ (TGF-β₁) family of proteins has the most potent stimulatory effect on extracellular matrix deposition of any cytokines so far examined. In animal models of pulmonary fibrosis enhanced TGF-β₁ gene expression is temporally and spatially related to increased collagen gene expression and protein deposition. Several lines of evidence suggest that TGF-β is a central regulator of pul-monary fibrosis. Several animal models over expressing TGF-β showed extensive progressive fibrosis but limited inflammation, indicating that TGF-β may play a predominant role in the progression of pulmonary fibrosis.

Diseases involving the lung associated with increased levels of TGF-β include rapid progressive pulmonary fibrosis, giant-cell interstitial pneumonia, acute rejection after lung transplantation, viral pneumonitis, chronic obstructive lung disease, and asthma.

### Increased TNF Levels in Fibrotic Diseases

An important role of TNF in interstitial fibrosis has been established using transgenic mice, which either overexpress or display a deficiency of this cytokine. Mice transgenically modified to overexpress TNF develop lung fibrosis. In contrast, mice null for TNF shows marked resistance to bleomycin induced fibrosis. TNF can stimulate fibroblast replication and collagen synthesis in vitro, and pulmonary TNF gene expression rises after administration of bleomycin in mice. Soluble TNF receptors reduce lung fibrosis in murine models and pulmonary overexpression of TNF in transgenic mice is characterized by lung fibrosis. In patients with CFA or asbestosis, bronchoalveolar lavage fluid-derived macrophages release increased amounts of TNF compared with controls.

Pulmonary fibrosis can be an all too common consequence of an acute inflammatory response of the lung to a host of inciting events. Chronic lung injury due to fibrotic changes can result from an identifiable inflammatory event or an insidious, unknown event. The inflammatory process can include infiltration of various inflammatory cell types, such as neutrophils and macrophages, the secretion of inflammatory cytokines and chemokines and the secretion of matrix remodelling proteinases.

### Inflammatory Disease

Inflammation is the final common pathway of various insults, such as infection, trauma, and allergies to the human body. It is characterized by activation of the immune system with recruitment of inflammatory cells, production of pro-inflammatory cells and production of pro-inflammatory cytokines. Most inflammatory diseases and disorders are characterized by abnormal accumulation of inflammatory cells including monocytes/macrophages, granulocytes, plasma cells, lymphocytes and platelets. Along with tissue endothelial cells and fibroblasts, these inflammatory cells release a complex array of lipids, growth factors, cytokines and destructive enzymes that cause local tissue damage.

One form of inflammatory response is neutrophilic inflammation which is characterized by infiltration of the inflamed tissue by neutrophil polymorphonuclear leukocytes (PMN), which are a major component of the host defense. Tissue infection by extracellular bacteria represents the prototype of this inflammatory response. On the other hand, various non-infectious diseases are characterized by extravascular recruitment of neutrophils. This group of inflammatory diseases includes chronic obstructive pulmonary disease, adult respiratory distress syndrome, some types of immune-complex alveolitis, cystic fibrosis, bronchitis, bronchiectasis, emphysema, glomerulonephritis, rheumatoid arthritis, gouty arthritis, ulcerative colitis, certain dermatoses such as psoriasis and vasculitis. In these conditions neutrophils are thought to play a crucial role in the development of tissue injury which, when persistent, can lead to the irreversible destruction of the normal tissue architecture with consequent organ dysfunction. Tissue damage is primarily caused by the activation of neutrophils followed by their release of proteinases and increased production of oxygen species.

Symptoms and signs of inflammation associated with specific conditions include:
rheumatoid arthritis:—pain, swelling, warmth and tenderness of the involved joints; generalized and morning stiffness;
insulin-dependent diabetes mellitus-insulitis; this condition can lead to a variety of complications with an inflammatory component, including:—retinopathy, neuropathy, nephropathy; coronary artery disease, peripheral vascular disease, and cerebrovascular disease;
autoimmune thyroiditis:—weakness, constipation, shortness of breath, puffiness of the face, hands and feet, peripheral edema, bradycardia;
multiple sclerosis:—spasticity, blurry vision, vertigo, limb weakness, paresthesias;
uveoretinitis:—decreased night vision, loss of peripheral vision;
lupus erythematosus:—joint pain, rash, photosensitivity, fever, muscle pain, puffiness of the hands and feet, abnormal urinalysis (haematuria, cylinduria, proteinuria), glomerulonephritis, cognitive dysfunction, vessel thrombosis, pericarditis;
scleroderma:—Raynaud's disease; swelling of the hands, arms, legs and face; skin thickening; pain, swelling and stiffness of the fingers and knees, gastrointestinal dysfunction, restrictive lung disease; pericarditis; renal failure;
other arthritic conditions having an inflammatory component such as rheumatoid spondylitis, osteoarthritis, septic arthritis and polyarthritis:—fever, pain, swelling, tenderness;
other inflammatory brain disorders, such as meningitis, Alzheimer's disease, AIDS dementia encephalitis:—photophobia, cognitive dysfunction, memory loss;
other inflammatory eye inflammations, such as retinitis:—decreased visual acuity;
inflammatory skin disorders, such as, eczema, other dermatitis (e.g., atopic, contact), psoriasis, burns induced by UV radiation (sun rays and similar UV sources):—erythema, pain, scaling, swelling, tenderness;
inflammatory bowel disease, such as Crohn's disease, ulcerative colitis:—pain, diarrhoea, constipation, rectal bleeding, fever, arthritis;
asthma:—shortness of breath, wheezing;
other allergy disorders, such as allergic rhinitis:—sneezing, itching, runny nose
conditions associated with acute trauma such as cerebral injury following stroke-sensory loss, motor loss, cognitive loss;
heart tissue injury due to myocardial ischemia:—pain, shortness of breath;
lung injury such as that which occurs in adult respiratory distress syndrome:—shortness of breath, hyperventilation, decreased oxygenation, pulmonary infiltrates;
inflammation accompanying infection, such as sepsis, septic shock, toxic shock syndrome:—fever, respiratory failure, tachycardia, hypotension, leukocytosis;
other inflammatory conditions associated with particular organs or tissues, such as:
   (i) nephritis (e.g., glomerulonephritis):—oliguria, abnormal urinalysis;
   (ii) inflamed appendix:—fever, pain, tenderness, leukocytosis;
   (iii) gout:—pain, tenderness, swelling and erythema of the involved joint, elevated serum and/or urinary uric acid;
   (iv) inflamed gall bladder:—abdominal pain and tenderness, fever, nausea, leukocytosis;
   (v) congestive heart failure:—shortness of breath, rales, peripheral edema;
   (vi) Type II diabetes:—end organ complications including cardiovascular, ocular, renal, and peripheral vascular disease;
   (vii) lung (pulmonary) fibrosis:—hyperventilation, shortness of breath, decreased oxygenation;
   (viii) vascular disease, such as atherosclerosis and restenosis:—pain, loss of sensation, diminished pulses, loss of function; and
   (ix) alloimmunity leading to transplant rejection:—pain, tenderness, fever.

T lymphocytes are a major source of cytokines. These cells bear antigen specific receptors on their cell surface to allow recognition of foreign pathogens. They can also recognize normal tissue during episodes of autoimmune diseases. There are two main subsets of T lymphocytes, distinguished by the presence of cell surface molecules known as CD4 and CD8. T lymphocytes expressing CD4 are also known as helper T cells, and these are regarded as being the most prolific cytokine producers. This subset can be further subdivided into Th1 and Th2, and the cytokines they produce are known as Th1-type cytokines and Th2-type cytokines.

Th1-type cytokines tend to produce the pro-inflammatory responses responsible for killing intracellular parasites and for perpetuating autoimmune responses. Interferon gamma is the main Th1 cytokine. Excessive pro-inflammatory responses can lead to uncontrolled tissue damage, so there needs to be a mechanism to counteract this. The Th2-type cytokines include Interleukin 4, Interleukin 5, and Interleukin 13, which are associated with the promotion of IgE and eosinophilic responses in atopy, and also Interleukin-10, which has more of an anti-inflammatory response. In excess, Th2 responses will counteract the Th1 mediated microbicidal action. The optimal scenario would therefore seem to be that humans should produce a well-balanced Th1 and Th2 response, suited to the immune challenge.

Allergy is regarded as a Th2 weighted imbalance. Pregnancy and early postnatal life are also viewed as Th2 phenomena (to reduce the risk of miscarriage, a strong Th2 response is necessary to modify the Th1 cellular response in utero). The foetus can switch on an immune response early in pregnancy, and because pregnancy is chiefly a Th2 situation, babies tend to be born with Th2 biased immune responses. These can be switched off rapidly postnatally under the influence of microbiological exposure or can be enhanced by early exposure to allergens.

Th1 cells produce interferon-gamma, Interleukin 2, and tumor necrosis factor-beta, which activate macrophages and are responsible for cell-mediated immunity and phagocyte-dependent protective responses. By contrast, Th2 cells produce cytokines which are responsible for strong antibody production, eosinophil activation, and inhibition of several macrophage functions, thus providing phagocyte-independent protective responses. Th1 cells mainly develop following infections by intracellular bacteria and some viruses, whereas Th2 cells predominate in response to infestations by gastrointestinal nematodes. Polarized Th1 and Th2 cells not only exhibit different functional properties, but also show the preferential expression of some activation markers and distinct transcription factors. Several mechanisms may influence the Th cell differentiation, which include the cytokine profile of “natural immunity” evoked by different offending agents, as well as the activity of some costimulatory molecules and microenvironmentally secreted hormones, in the context of the individual genetic background. In addition to playing different roles in protection, polarized Th1-type and Th2-type responses are also responsible for different types of immunopathological reactions. Th1 cells are involved in the pathogenesis of organ-specific autoimmune disorders like Crohn's disease is one, Helicobacter pylori-induced peptic ulcer, acute kidney allograft rejection, unexplained recurrent abortions, tuberculosis, myocarditis multiple sclerosis, scleroderma, Type 1 diabetes, rheumatoid arthritis (RA), sarcoidosis, autoimmune thyroiditis and uveitis. In contrast, allergen-specific Th2 responses are responsible for atopic disorders in genetically susceptible individuals like asthma. Moreover, Th2 responses against still unknown antigens predominate in Omenn's syndrome, idiopathic pulmonary fibrosis, and progressive systemic sclerosis. Finally, the prevalence of Th2 responses may play some role in a more rapid evolution of human immunodeficiency virus infection to the full-blown disease.

It is object of current application to find medications that produce a well-balanced Th1 and Th2 response in situations of Th1 predominance, in the situation of Th2 predominance, or even at situations where both, Th1 and Th2 cells are too much activated, as exemplified after severe infections.

Infections in Humans Associated with Autoimmune Diseases

| Disease | Infection |
|---|---|
| Multiple sclerosis | Epstein-Barr virus (EBV), measles virus |
| Lyme arthritis | Borrelia burgdorferi |
| Type I diabetes | Coxsackie virus B4, rubella virus, |
| | cytomegalovirus (CMV), mumps virus |
| Rheumatoid arthritis | Escherichia coli, mycobacteria, EBV, |
| | hepatitis C virus (HCV) |
| Lupus erythematosus | EBV |
| Myocarditis | CB3, CMV, chlamydia |
| Rheumatic fever/myocarditis | Streptococci |
| Chagas' disease/myocarditis | Trypanosoma cruzi |
| Myasthenia gravis | Herpes simplex virus, HCV |

### Neurodegenerative Disease

The present invention also relates generally to the fields of neurology and to methods of protecting the cells of a mammalian central nervous system from infection damage or injury.

Injuries or trauma of various kinds to the central nervous system (CNS) or the peripheral nervous system (PNS) can produce profound and long-lasting neurological and/or psychiatric symptoms and disorders. One form that this can take is the progressive death of neurons or other cells of the central nervous system (CNS), i.e., neurodegeneration or neuronal degeneration.

Patients with injury or damage of any kind to the central (CNS) or peripheral (PNS) nervous system including the retina may benefit from neuroprotective methods. This nervous system injury may take the form of an abrupt insult or an acute injury to the nervous system as in, for example, acute neurodegenerative disorders including, but not limited to; acute injury, hypoxia-ischemia or the combination thereof resulting in neuronal cell death or compromise.

In addition, deprivation of oxygen or blood supply in general can cause acute injury as in hypoxia and/or ischemia including, but not limited to, cerebrovascular insufficiency, cerebral ischemia or cerebral infarction (including cerebral ischemia or infarctions originating from embolic occlusion and thrombosis, retinal ischemia (diabetic or otherwise), glaucoma, retinal degeneration, multiple sclerosis, toxic and ischemic optic neuropathy, reperfusion following acute ischemia, perinatal hypoxic-ischemic injury, cardiac arrest or intracranial haemorrhage of any type (including, but not limited to, epidural, subdural, subarachnoid or intracerebral haemorrhage).

Other disorders associated with neuronal injury include, but are not limited to, disorders associated with chemical, toxic, infectious and radiation injury of the nervous system including the retina, or those neuropathies originating from infections, inflammation, immune disorders, drug abuse, pharmacological treatments, toxins, or trauma.

Further indications are cognitive disorders. The term “cognitive disorder” shall refer to anxiety disorders, delirium, dementia, amnestic disorders, dissociative disorders, eating disorders, mood disorders, sleep disorders, or acute stress disorder.

The term “neuroprotection” as used herein shall mean; inhibiting, preventing, ameliorating or reducing the severity of the dysfunction, degeneration or death of nerve cells, axons or their supporting cells in the central or peripheral nervous system of a mammal, including a human. This includes the treatment or prophylaxis of a neurodegenerative disease; protection against excitotoxicity or ameliorating the cytotoxic effect of an infection in a patient in need thereof.

The term “a patient in need of treatment with a neuroprotective drug” as used herein will refer to any patient who currently has or may develop any of the above syndromes or disorders, or any disorder in which the patient's present clinical condition or prognosis could benefit from providing neuroprotection to prevent the development, extension, worsening or increased resistance to treatment of any neurological or psychiatric disorder.

Thus, in some embodiments, the means and methods of the present invention are directed toward neuroprotection in a subject who is at risk of developing neuronal damage but who has not yet developed clinical evidence. This patient may simply be at “greater risk” as determined by the recognition of any factor in a subject's, or their families, medical history, physical exam or testing that is indicative of a greater than average risk for developing neuronal damage. Therefore, this determination that a patient may be at a “greater risk” by any available means can be used to determine whether the patient should be treated with the methods of the present invention.

Accordingly, in an exemplary embodiment, subjects who may benefit from treatment by the methods and medications of this invention can be identified using accepted screening methods to determine risk factors for neuronal damage. These screening methods include, for example, conventional work-ups to determine risk factors including but not limited to: for example, CNS infections, bacterial or viral.

It is expected that many more such biomarkers utilizing a wide variety of detection techniques will be developed in the future. It is intended that any such marker or indicator of the existence or possible future development of neuronal damage, as the latter term is used herein, may be used in the methods of this invention for determining the need for treatment with the compounds and methods of this invention.

As used herein the term “combination administration” of a compound, therapeutic agent or known drug with a medication of the present invention means administration of the drug and the one or more compounds at such time that both the known drug and the medication will have a therapeutic effect. In some cases, this therapeutic effect will be synergistic. Such concomitant administration can involve concurrent (i.e., at the same time), prior, or subsequent administration of the drug with respect to the administration of a medication of the present invention. A person of ordinary skill in the art would have no difficulty determining the appropriate timing, sequence and dosages of administration for particular drugs and medications of the present invention.

### Heart and Vascular Disease

Heart disease is a general term used to describe many different heart conditions. For example, coronary artery disease, which is the most common heart disease, is characterized by constriction or narrowing of the arteries supplying the heart with oxygen-rich blood, and can lead to myocardial infarction, which is the death of a portion of the heart muscle. Heart failure is a condition resulting from the inability of the heart to pump an adequate amount of blood through the body. Heart failure is not a sudden, abrupt stop of heart activity but, rather, typically develops slowly over many years, as the heart gradually loses its ability to pump blood efficiently.

Vascular diseases are often the result of decreased perfusion in the vascular system or physical or biochemical injury to the blood vessel.

Peripheral vascular disease (PVD) is defined as a disease of blood vessels often encountered as narrowing of the vessels of the limbs. There are two main types of these disorders, functional disease which doesn't involve defects in the blood vessels but rather arises from stimuli such as cold, stress, or smoking, and organic disease which arises from structural defects in the vasculature such as atherosclerotic lesions, local inflammation, or traumatic injury. This can lead to occlusion of the vessel, aberrant blood flow, and ultimately to tissue ischemia.

One of the more clinically significant forms of PVD is peripheral artery disease (PAD). PAD is often treated by angioplasty and implantation of a stent or by artery bypass surgery. Clinical presentation depends on the location of the occluded vessel. For example, narrowing of the artery that supplies blood to the intestine can result in severe postprandial pain in the lower abdomen resulting from the inability of the occluded vessel to meet the increased oxygen demand arising from digestive and absorptive processes. In severe forms the ischemia can lead to intestinal necrosis. Similarly, PAD in the leg can lead to intermittent pain, usually in the calf, that comes and goes with activity. This disorder is known as intermittent claudication (IC) and can progress to persistent pain while resting, ischemic ulceration, and even amputation.

Peripheral vascular disease is also manifested in atherosclerotic stenosis of the renal artery, which can lead to renal ischemia and kidney dysfunction.

One disease in which vascular diseases and their complications are very common is diabetes mellitus. Diabetes mellitus causes a variety of physiological and anatomical irregularities, the most prominent of which is the inability of the body to utilize glucose normally, which results in hyperglycemia. Chronic diabetes can lead to complications of the vascular system which include atherosclerosis, abnormalities involving large and medium size blood vessels (macroangiopathy) and abnormalities involving small blood vessels (microangiopathy) such as arterioles and capillaries.

In addition to large vessel disease, patients with diabetes suffer further threat to their skin perfusion in at least two additional ways. First, by involvement of the non-conduit arteries, which are detrimentally affected by the process of atherosclerosis, and secondly, and perhaps more importantly, by impairment of the microcirculatory control mechanisms (small vessel disease). Normally, when a body part suffers some form of trauma, the body part will, as part of the body's healing mechanism, experience an increased blood flow. When small vessel disease and ischemia are both present, as in the case of many diabetics, this natural increased blood flow response is significantly reduced. This fact, together with the tendency of diabetics to form blood clots (thrombosis) in the microcirculatory system during low levels of blood flow, is believed to be an important factor in ulcer pathogenesis.

Other diseases, although not known to be related to diabetes are similar in their physiological effects on the peripheral vascular system. Such diseases include Raynaud syndrome, CREST syndrome, autoimmune diseases such as lupus erythematosus, rheumatoid disease, and the like.

Accordingly, the invention is at least in part based on the surprising finding, that the means and methods of the invention can be used in the prevention and/or treatment of disease or disorder secondary to the viral infection.

### Pharmaceutical Compositions

Still another aspect of the present invention relates to the use of a combination of two active compounds as active ingredients, together with at least one pharmaceutically acceptable carrier, excipient and/or diluents for the manufacture of a pharmaceutical composition for the treatment and/or prophylaxis of an infectious disease, an autoimmune disease, a fibrotic disease, an inflammatory disease, a neurodegenerative disease, or a heart and vascular disease as a consequence of a viral disease such as a COVID-19 disease.

Such pharmaceutical compositions comprise a combination of two active compounds as active ingredients, together with at least one pharmaceutically acceptable carrier, excipient, binders, disintegrates, glidants, diluents, lubricants, colouring agents, sweetening agents, flavouring agents, preservatives or the like. The pharmaceutical compositions of the present invention can be prepared in a conventional solid or liquid carrier or diluents and a conventional pharmaceutically-made adjuvant at suitable dosage level in a known way.

Preferably the combination of two active compounds is suitable for intravenous administration or suitable for oral administration or suitable for administration by inhalation.

Any aerosol formulation approach designed to deliver substances to the lungs must ultimately produce a respirable aerosol. In general, two different options exist: a nebulizer and a pressurized metered-dose inhaler (MDI), preferably as a dry powder inhaler.

MDIs are pressurized canisters containing a mixture of propellants, surfactants, preservatives, and flavour agents, a metering valve, for metering the dispensed quantity and a mouthpiece for inhaling. When the actuator is depressed, the mixture is released from the canister through a metering valve and stem. MDIs are widely in use, e.g., in the treatment of asthma.

The pharmaceutical formulation consists of the drug, a liquefied gas propellant such as hydrofluoroalkanes and optionally pharmaceutically acceptable excipients.

The water content is a critical issue in MDI in the actuator and spacer deposition, leading to a decrease of fine particle fraction, which in turn is an issue for deep lung deposition in the patient. Therefore, a dry powder MDI is preferred. Dry powder inhalers (DPI) deliver the drug to the lungs in the form of a dry powder. Most DPIs rely on the force of patient inhalation to entrain powder from the device and subsequently break-up the powder into particles that are small enough to reach the lungs. For this reason, insufficient patient inhalation flow rates may lead to reduced dose delivery and incomplete disaggregation of the powder, leading to unsatisfactory device performance. Thus, most DPIs need a minimum inspiratory effort for proper use. Therefore, their use is limited to older children and adults.

While disorders affecting the bronchi or upper parts of the lower airways can be addressed this way, e.g., by asthma sprays, disorders affecting the alveoli where the gas exchange takes place can be only insufficiently treated because of ineffective inhalatory administration, e.g., in COPD. The administered drug particles are not able to reach the bottom of the lungs by way of inhalation, at least not in a therapeutically effective amount.

Nebulizers use to administer the active principle in the form of a mist inhaled into the lungs. Physically, this mist is an aerosol. It is generated in the nebulizer by breaking up solutions and suspensions into small aerosol droplets (preferred) or solid particles that can be directly inhaled from the mouthpiece of the device. In conventional nebulizers the aerosol can be generated by mechanical force, e.g., spring force in soft mist nebulizers, or electrical force. In jet nebulizers a compressor brings oxygen or compressed air to flow at high velocity through the aqueous solution with the active principle, this way generating an aerosol.

Ultrasonic wave nebulizers use an electronic oscillator that at high frequency causes vibration of a piezoelectric element for generating ultrasonic waves in the liquid reservoir with the active principle.

The most promising technology are vibrating mesh nebulizers. They use a mesh, respectively a polymer membrane having a very large number of laser-drilled holes. This membrane is placed between the liquid reservoir and the aerosol chamber. A piezoelectric element placed on the membrane induces high frequency vibrations of the membrane, leading to droplet formation in the aqueous solution and pressuring these droplets through the holes of the membrane into the aerosol chamber. With this technique very small droplet sizes can be generated. Moreover, a significantly shorter inhalation time for the patient can thus be achieved, a feature which drastically increases patient compliance. Only these mesh nebulizers are regarded to be able to generate liquid droplets with the active principle in the desired size range and bring them in a therapeutically effective amount into the patient's alveoli in a reasonable time.

In certain embodiments, the invention relates to the Aviptadil, functional derivative or precursor for use of the invention, wherein the post-viral infection syndrome is post-COVID-19 syndrome.

In certain embodiments, the invention relates to the vector for use of the invention, wherein the post-viral infection syndrome is post-COVID-19 syndrome.

In certain embodiments, the invention relates to the biological cell for use of the invention, wherein the post-viral infection syndrome is post-COVID-19 syndrome.

In certain embodiments, the invention relates to the composition for use of the invention, wherein the post-viral infection syndrome is post-COVID-19 syndrome.

In certain embodiments, the invention relates to the pharmaceutical product for use of the invention, wherein the post-viral infection syndrome is post-COVID-19 syndrome.

The term “post-COVID-19 syndrome”, as used herein, refers to a post viral syndrome, wherein the viral infection is a SARS-COV-2 infection. In some embodiments, the SARS-COV-2 described herein is a SARS-COV-2 variant selected from the group consisting of Lineage B.1, Lineage B.1.1.207, Lineage B.1.1.7, Cluster 5, 501. V2 variant, Lineage P.1, Lineage B.1.429/CAL.20C, Lineage B.1.427, Lineage B.1.526, Lineage B.1.525, Lineage B.1.1.317, Lineage B.1.1.318, Lineage B.1.351, Lineage B.1.617, B.1.617.1, B.1.617.2, B.1.617.3, B.1.618, and Lineage P.3. In some aspects, the SARS-CoV-2 described herein is a SARS-COV-2 variant described by a Nextstrain clade selected from the group consisting of 19A, 20A, 20C, 20G, 20H, 20B, 20D, 20F, 20I, and 20E. In some aspects, the SARS-COV-2 described herein is a SARS-COV-2 variant comprising at least one mutation selected from the group consisting of D614G, E484K, N501Y, S477G/N, P681H, E484Q, L452R and P614R. In some aspects, the SARS-COV-2 described herein is a SARS-COV-2 variant derived from the variants described herein. In some aspects, the SARS-COV-2 described herein is a SARS-COV-2 variant having an at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% sequence identity to the viral genome sequence of at last one SARS-COV-2 variant described herein.

Post-COVID-19 syndrome is a poorly understood syndrome. The means and methods provided herein have shown to be effective in treating patients suffering from the disorder (Example 1-4).

Accordingly, the invention is at least in part based on the surprising finding, that the means and methods described herein are effective in the treatment of post-COVID-19 syndrome.

In certain embodiments, the invention relates to Aviptadil, a functional derivative or precursor for use of the invention for use in treatment of at least one symptom of post-COVID-19 syndrome selected from the group consisting of fatigue, somnolence, headaches, dizziness, cerebrovascular disease, seizures, neuropathy and encelopathy.

In certain embodiments, the invention relates to the vector for use of the invention for use in treatment of at least one symptom of post-COVID-19 syndrome selected from the group consisting of fatigue, somnolence, headaches, dizziness, cerebrovascular disease, seizures, neuropathy and encelopathy.

In certain embodiments, the invention relates to the biological cell for use of the invention for use in treatment of at least one symptom of post-COVID-19 syndrome selected from the group consisting of fatigue, somnolence, headaches, dizziness, cerebrovascular disease, seizures, neuropathy and encelopathy.

In certain embodiments, the invention relates to the composition for use of the invention for use in treatment of at least one symptom of post-COVID-19 syndrome selected from the group consisting of fatigue, somnolence, headaches, dizziness, cerebrovascular disease, seizures, neuropathy and encelopathy.

In certain embodiments, the invention relates to the pharmaceutical product for use of the invention for use in treatment of at least one symptom of post-COVID-19 syndrome selected from the group consisting of fatigue, somnolence, headaches, dizziness, cerebrovascular disease, seizures, neuropathy and encelopathy.

In one aspect, methods and therapeutic medications are provided for treating such conditions as post COVID-19 syndrome, especially Fatigue, Somnolence, Headaches, Dizziness, Cerebrovascular disease, Seizures, Neuropathy, Encephalopathy by administering to a subject in need thereof an intranasal formulation of Aviptadil.

The means and methods described herein can be enabled for delivery of the therapeutics described herein to the nervous system, e.g. across the blood brain barrier.

Accordingly, the invention is at least in part based on the surprising finding, that the means and methods described herein are effective in the treatment symptoms of post-COVID-19 syndrome relating to the nervous system.

In certain embodiments, the invention relates to Aviptadil, functional derivative or precursor for use of the invention, for use in oral, intravenous, inhalation and/or intranasal treatment.

In certain embodiments, the invention relates to the vector for use of the invention for use in oral, intravenous, inhalation and/or intranasal treatment.

In certain embodiments, the invention relates to the biological cell for use of the invention for use in oral, intravenous, inhalation and/or intranasal treatment.

In certain embodiments, the invention relates to the composition for use of the invention for use in oral, intravenous, inhalation and/or intranasal treatment.

In certain embodiments, the invention relates to the pharmaceutical product for use of the invention, for use in oral, intravenous, inhalation and/or intranasal treatment.

In certain embodiments, the invention relates to Aviptadil, functional derivative or precursor for use of the invention, for use in intranasal treatment.

In certain embodiments, the invention relates to the vector for use of the invention for use in intranasal treatment.

In certain embodiments, the invention relates to the biological cell for use of the invention for use in intranasal treatment.

In certain embodiments, the invention relates to the composition for use of the invention for use in intranasal treatment.

In certain embodiments, the invention relates to the pharmaceutical product for use of the invention, for use in intranasal treatment.

In certain embodiments, the invention relates to Aviptadil, functional derivative or precursor for use of the invention, the vector for use of the invention, the biological cell for use of the invention, the composition for use of the invention or the pharmaceutical product for use of the invention, wherein the intranasal administration is direct nose-to-brain administration.

In certain embodiments, the invention relates to the vector for use of the invention wherein the intranasal administration is direct nose-to-brain administration.

In certain embodiments, the invention relates to the biological cell for use of the invention, wherein the intranasal administration is direct nose-to-brain administration.

In certain embodiments, the invention relates to the composition for use of the invention, wherein the intranasal administration is direct nose-to-brain administration.

In certain embodiments, the invention relates to the pharmaceutical product for use of the invention, wherein the intranasal administration is direct nose-to-brain administration.

In some embodiments, delivery of the intranasal formulation is by traditional intranasal delivery wherein a formulation is sprayed on or deposited on the respiratory area within the nasal cavity. In some embodiments, delivery of the intranasal formulation is direct “nose-to-brain” delivery wherein the formulation is contacted with the olfactory area within the nasal cavity, enabling transport of compounds directly into the brain via olfactory neurons. By way of non-limiting example, Aviptadil is administered intranasally twice a day.

In some embodiments, the dose and dosing regimen of Aviptadil, provides relief from symptoms such as post COVID-19 syndrome, especially Fatigue, Somnolence, Headaches, Dizziness, Cerebrovascular disease, Seizures, Neuropathy, Encephalopathy. Not only are there no medications approved for post COVID-19 syndrome, nor a medication for post COVID-19 syndrome has been evaluated in double-blind, placebo-controlled trials. Furthermore, no medication post COVID-19 syndrome has been evaluated in ‘nose-to-brain’ intranasal delivery systems.

### Intranasal Formulations and Delivery Devices

Aviptadil may be used in an intranasal formulation and delivery system for the treatment of conditions and diseases such as and including those described herein.

In one aspect, intranasal delivery is via direct (also called deep) nose-to-brain delivery, i.e., to the olfactory region of the nasal cavity. In one aspect, intranasal delivery is via traditional nasal delivery, i.e., to the respiratory area of the nasal cavity. Direct “nose-to-brain” delivery of Aviptadil may be achieved by use of any one of several methods that comprise a nasal formulation and/or a nasal delivery device to deliver Aviptadil to the roof of the nasal cavity, where transport into the central nervous system (CNS) is achieved. Nose-to-brain delivery is a minimally invasive drug administration pathway, which bypasses the blood-brain barrier as the drug is directed from the nasal cavity to the brain. In particular, the skull base located at the roof of the nasal cavity is in close vicinity to the CNS. This area is covered with olfactory mucosa. To design and tailor suitable formulations for nose-to-brain drug delivery, the architecture, structure and physicochemical characteristics of the mucosa are important criteria. See, for example, Gänger et al., 2018, Tailoring Formulations for Intranasal Nose-to-Brain Delivery: A Review on Architecture, Physico-Chemical Characteristics and Mucociliary Clearance of the Nasal Olfactory Mucosa, Pharmaceutics. 2018 September; 10(3): 116; and Lalatsa et al., Strategies to deliver peptide drugs to the brain, Mol. Pharmaceuticas 2014, 11, 4, 1081-1093; Islam et al., 2020, Intranasal Delivery of Nanoformulations: A Potential Way of Treatment for Neurological Disorders, Molecules. 2020 April; 25(8): 1929; for formulations and/or devices for intranasal delivery, the foregoing of which are incorporated herein by reference.

### Intranasal Formulations

Aviptadil as described above may be provided for administration in any formulation compatible with intranasal administration including direct nose-to-brain administration. The intranasal formulation may be a powder, a dry powder, a liquid, a gel, or any other form that achieves the intranasal delivery of Aviptadil as described herein. Post COVID-19 syndrome, especially Fatigue, Somnolence, Headaches, Dizziness, Cerebrovascular disease, Seizures, Neuropathy, Encephalopathy are among conditions and diseases treatable by Aviptadil, and wherein intranasal administration offers a facile and patient-compliant means for dosing.

Alkylglycosides and related compounds can be used to enhance intranasal delivery of peptides. U.S. Pat. No. 8,833,728 describes a pharmaceutical compositions and methods for delivering a peptide to the central nervous system of a mammal via intranasal administration. Nasal absorption is enhanced using, for example, an aqueous composition comprising a therapeutic peptide and a compound such as 1-O-n-dodecyl-beta-D-maltopyranoside, 1-O-n-decyl-beta-D-maltopyranoside, 1-O-n-tetradecyl-beta-D-maltopyranoside, beta-D-fructopyranosyl-alpha-glucopyranoside monododecanoate, or dodecyl-β-D-maltoside.

Administration forms include, for example, pills, tablets, film tablets, coated tablets, capsules, liposomal formulations, micro- and nano-formulations, powders and deposits. Furthermore, the present invention also includes pharmaceutical preparations for parenteral application, including dermal, intradermal, intragastric, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain a combination of two active compounds according to the present invention.

The combination of two active compounds of the invention act synergistically can also be administered in form of its pharmaceutically active salts.

Alpha Lipoic Acid (ALA) as an ingredient has been compounded for more than 25 years as an injection, suppository, topical and troche formulation. ALA is compounded for intravenous administration to treat diabetes and diabetic nephropathy. Available clinical reports revealed no serious safety concerns. However ALA is unstable in aqueous formulations, and has never been applied via inhalation. We have developed a solubilisated formulation of ALA that can also be used via inhalation to reach the deep lung of the patient.

Suitable pharmaceutically active salts comprise acid addition salts and alkali or earth alkali salts. For instance, sodium, potassium, lithium, magnesium or calcium salts can be obtained.

The combination of two active compounds of the invention forms pharmaceutically acceptable salts with organic and inorganic acids. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphersulfonic acid, china acid, mandelic acid, o-methylmandelic acid, hydrogen-benzenesulfonic acid, picric acid, adipic acid, D-o-tolyltartaric acid, tartronic acid, toluic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner.

The pharmaceutical compositions according to the present invention will typically be administered together with suitable carrier materials selected with respect to the intended form of administration, i.e., for oral administration in the form of tablets, capsules (either solid filled, semi-solid filled or liquid filled), powders for constitution, aerosol preparations consistent with conventional pharmaceutical practices. Other suitable formulations are gels, elixirs, dispersible granules, syrups, suspensions, creams, lotions, solutions, emulsions, suspensions, dispersions, and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

As pharmaceutically acceptable carrier, excipient and/or diluents can be used lactose, starch, sucrose, cellulose, magnesium stearate, dicalcium phosphate, calcium sulphate, talc, mannitol, ethyl alcohol (liquid filled capsules).

Suitable binders include starch, gelatine, natural sugars, corn sweeteners, natural and synthetic gums such as acacia, sodium alginate, carboxymethyl-cellulose, polyethylene glycol and waxes. Among the lubricants that may be mentioned for use in these dosage forms, boric acid, sodium benzoate, sodium acetate, sodium chloride, and the like. Disintegrants include starch, methylcellulose, guar gum and the like. Sweetening and flavouring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate-controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example, may be mentioned water or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g., nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized moulds, allowed to cool and thereby solidify.

Also included are solid form preparations which are intended to be converted, shortly before use, to liquid form preparations for either oral or parenteral administration. Such liquid forms include solutions, suspensions and emulsions.

The term capsule refers to a special container or enclosure made of methyl cellulose, polyvinyl alcohols, or denatured gelatines or starch for holding or containing compositions comprising the active ingredients. Hard shell capsules are typically made of blends of relatively high gel strength bone and pork skin gelatines. The capsule itself may contain small amounts of dyes, opaquing agents, plasticizers and preservatives.

Tablet means compressed or moulded solid dosage form containing the active ingredients with suitable diluents. The tablet can be prepared by compression of mixtures or granulations obtained by wet granulation, dry granulation or by compaction well known to a person skilled in the art.

Binders characterize substances that bind or “glue” powders together and make them cohesive by forming granules, thus serving as the “adhesive” in the formulation. Binders add cohesive strength already available in the diluents or bulking agent. Suitable binders include sugars such as sucrose, starches derived from wheat, corn rice and potato; natural gums such as acacia, gelatine and tragacanth; derivatives of seaweed such as alginic acid, sodium alginate and ammonium calcium alginate; cellulosic materials such as methylcellulose and sodium carboxymethylcellulose and hydroxypropyl-methylcellulose; polyvinylpyrrolidone; and inorganics such as magnesium aluminium silicate. The amount of binder in the composition can range from about 1 to 30% by weight of the composition, preferably from about 2 to about 20% by weight of the composition, more preferably from about 3 to about 10% by weight, even more preferably from about 3 to about 6% by weight.

Lubricant refers to a substance added to the dosage form to enable the tablet, granules, etc. after it has been compressed, to release from the mould or die by reducing friction or wear. Suitable lubricants include metallic stearates such as magnesium stearate, calcium stearate or potassium stearate; stearic acid; high melting point waxes; and water-soluble lubricants such as sodium chloride, sodium benzoate, sodium acetate, sodium oleate, polyethylene glycols and DL-leucine. Lubricants are usually added at the very last step before compression, since they must be present on the surfaces of the granules and in between them and the parts of the tablet press. The amount of lubricant in the composition can range from about 0.05 to about 15% by weight of the composition, preferably 0.2 to about 5% by weight of the composition, more preferably from about 0.3 to about 3%, and most preferably from about 0.3 to about 1.5% by weight of the composition.

Glidants are materials that prevent caking and improve the flow characteristics of granulations, so that flow is smooth and uniform. Suitable glidants include silicon dioxide and talc. The amount of glidant in the composition can range from about 0.01 to 10% by weight of the composition, preferably 0.1% to about 7% by weight of the total composition, more preferably from about 0.2 to 5% by weight, and most preferably from about 0.5 to about 2% by weight.

Colouring agents are excipients that provide coloration to the composition or the dosage form. Such excipients can include food grade dyes and food grade dyes adsorbed onto a suitable adsorbent such as clay or aluminium oxide. The amount of the colouring agent can vary from about 0.01 to 10% by weight of the composition, preferably from about 0.05 to 6% by weight, more preferably from about 0.1 to about 4% by weight of the composition, and most preferably from about 0.1 to about 1%.

The term “buffer,” when used with reference to hydrogen-ion concentration or pH, refers to the ability of a system, particularly an aqueous solution, to resist a change of pH on adding acid or alkali, or on dilution with a solvent. Preferred buffers can be selected from the group consisting of formate (pKa=3.75), lactate (pKa=3.86), benzoic acid (pKa-4.2) oxalate (pKa=4.29), fumarate (pKa=4.38), aniline (pKa=4.63), acetate buffer (pKa-4.76), citrate buffer (pKa2-4.76, pKa3-6.4), glutamate buffer (pKa=4.3), phosphate buffer (pKa=7.20), succinate (pKa1-4.93; pKa2-5.62), pyridine (pKa=5.23), phthalate (pKa=5.41); histidine (pKa-6.04), MES (2-(N-morpholino)ethanesulphonic acid; pKa=6.15); maleic acid (pKa=6.26); cacodylate (dimethylarsinate, pKa-6.27), carbonic acid (pKa-6.35), ADA (N-(2-acetamido)imino-diacetic acid (pKa-6.62); PIPES (4-piperazinebis-(ethanesulfonic acid; BIS-TRIS-propane (1,3-bis[tris(hydroxymethyl)methylamino]-propane), pKa-6.80), ethylendiamine (pKa=6.85), ACES 2-[(2-amino-2-oxoethyl)amino]ethanesulphonic acid; pKa-6.9), imidazole (pKa=6.95), MOPS (3-(N-morphin)-propansulfonic acid; pKa-7.20), diethylmalonic acid (pKa=7.2), TES (2-[tris (hydroxymethyl) methyl] amino ethanesulphonic acid; pKa=7.50) and HEPES (N-2-hydroxylethylpiperazin-N′-2-ethansulfonic acid; pKa=7.55) buffers or other buffers having a pKa between 3.8 to 7.7.

Preferred is the group of carboxylic acid buffers such as acetate and carboxylic diacid buffers such as fumarate, tartrate and phthalate and carboxylic triacid buffers such as citrate. Another group of preferred buffers is represented by inorganic buffers such as sulphate, borate, carbonate, oxalate, calcium hydroxide and phosphate buffers. Another group of preferred buffers are nitrogen containing buffers such as imidazole, diethylenediamine, and piperazine.

Also preferred are sulfonic acid buffers such as TES, HEPES, ACES, PIPES, [(2-hydroxy-1,1-bis(hydroxymethyl)ethyl)amino]-1-propanesulfonic acid (TAPS), 4-(2-hydroxyethyl)piperazine-1-propanesulfonic acid (EPPS), 4-Morpholinepropanesulfonic acid (MOPS) and N,N-bis(2-hydroxyethyl)-2-aminoethanesulfonic acid (BES).

Another group of preferred buffers are glycine buffers such as glycine, glycyl-glycine, glycyl-glycyl-glycine, N,N-bis(2-hydroxyethyl)glycine and N-[2-hydroxy-1,1-bis(hydroxy-methyl)ethyl]glycine (Tricine).

Preferred are also amino acid buffers such as glycine, alanine, valine, leucine, isoleucine, serine, threonine, phenylalanine, tyrosine, tryptophane, lysine, arginine, histidine, aspartate, glutamate, asparagine, glutamine, cysteine, methionine, proline, 4-hydroxyproline, N,N,N-trimethyllysine, 3-methylhistidine, 5-hydroxylysine, O-phosphoserine, γ-carboxyglutamate, ε-N-acetyllysine, ⋅-N-methylarginine, citrulline, ornithine and derivatives thereof.

Also preferred are the following buffers:

| effective pH range | pKa 25° C. | buffer |
|---|---|---|
| 2.7-4.2 | 3.40 | malate (pK1) |
| 3.0-4.5 | 3.75 | formate |
| 3.0-6.2 | 4.76 | citrate (pK2) |
| 3.2-5.2 | 4.21 | succinate (pK1) |
| 3.6-5.6 | 4.76 | acetate |
| 3.8-5.6 | 4.87 | propionate |
| 4.0-6.0 | 5.13 | malate (pK2) |
| 4.9-5.9 | 5.23 | pyridine |
| 5.0-6.0 | 5.33 | piperazine (pK1) |
| 5.0-7.4 | 6.27 | cacodylate |
| 5.5-6.5 | 5.64 | succinate (pK2) |
| 5.5-6.7 | 6.10 | MES |
| 5.5-7.2 | 6.40 | citrate (pK3) |
| 5.5-7.2 | 6.24 | maleate (pK2) |
| 5.5-7.4 | 1.70, 6.04, 9.09 | histidine |
| 5.8-7.2 | 6.46 | bis-tris |
| 5.8-8.0 | 7.20 | phosphate (pK2) |
| 6.0-12.0 | 9.50 | ethanolamine |
| 6.0-7.2 | 6.59 | ADA |
| 6.0-8.0 | 6.35 | carbonate (pK1) |
| 6.1-7.5 | 6.78 | ACES |
| 6.1-7.5 | 6.76 | PIPES |
| 6.2-7.6 | 6.87 | MOPSO |
| 6.2-7.8 | 6.95 | imidazole |
| 6.3-9.5 | 6.80, 9.00 | BIS-TRIS propane |
| 6.4-7.8 | 7.09 | BES |
| 6.5-7.9 | 7.14 | MOPS |
| 6.8-8.2 | 7.48 | HEPES |
| 6.8-8.2 | 7.40 | TES |
| 6.9-8.3 | 7.60 | MOBS |
| 7.0-8.2 | 7.52 | DIPSO |
| 7.0-8.2 | 7.61 | TAPSO |
| 7.0-8.3 | 7.76 | triethanolamine (TEA) |
| 7.0-9.0 | 0.91, 2.10, 6.70, 9.32 | pyrophosphate |
| 7.1-8.5 | 7.85 | HEPPSO |
| 7.2-8.5 | 7.78 | POPSO |

Preferred are the buffers having an effective pH range of from 2.7 to 8.5, and more preferred of from 3.8 to 7.7. The effective pH range for each buffer can be defined as pKa−1 to pKa+1, where Ka is the ionization constant for the weak acid in the buffer and pKa=−log K.

Most preferred are buffers suitable for pharmaceutical use e.g., buffers suitable for administration to a patient such as acetate, carbonate, citrate, fumarate, glutamate, lactate, phosphate, phthalate, and succinate buffers. Particularly preferred examples of commonly used pharmaceutical buffers are acetate buffer, citrate buffer, glutamate buffer and phosphate buffer. Also, most preferred is the group of carboxylic acid buffers. The term “carboxylic acid buffers” as used herein shall refer to carboxylic mono acid buffers and carboxylic diacid buffers as well as carboxylic triacid buffers. Of course, also combinations of buffers, especially of the buffers mentioned herein are useful for the present invention.

Some suitable pharmaceutical buffers are a citrate buffer (preferably at a final formulation concentration of from about 20 to 200 mM, more preferably at a final concentration of from about 30 to 120 mM) or an acetate buffer (preferably at a final formulation concentration of about 20 to 200 mM) or a phosphate buffer (preferably at a final formulation concentration of about 20 to 200 mM).

Techniques for the formulation and administration of the drugs of the present invention may be found in “Remington's Pharmaceutical Sciences” Mack Publishing Co., Easton PA. A suitable composition comprising at least one drug mentioned herein may be a solution of the drug in a suitable liquid pharmaceutical carrier or any other formulation such as tablets, pills, film tablets, coated tablets, dragees, capsules, powders and deposits, gels, syrups, slurries, suspensions, emulsions, and the like.

A particularly preferred pharmaceutical composition is a lyophilised (freeze-dried) preparation suitable for administration by inhalation or for intravenous administration. To prepare the preferred lyophilised preparation the combination of two active compounds of the invention is solubilised in a 4 to 5% (w/v) mannitol solution and the solution is then lyophilised. The mannitol solution can also be prepared in a suitable buffer solution as described above.

Further examples of suitable cryo-/lyoprotectants (otherwise referred to as bulking agents or stabilizers) include thiol-free albumin, immunoglobulins, polyalkyleneoxides (e.g., PEG, polypropylene glycols), trehalose, glucose, sucrose, sorbitol, dextran, maltose, raffinose, stachyose and other saccharides, while mannitol is used preferably. These can be used in conventional amounts in conventional lyophilization techniques. Methods of lyophilisation are well known in the art of preparing pharmaceutical formulations.

For administration by inhalation the particle diameter of the lyophilised preparation is preferably between 2 to 5 μm, more preferably between 3 to 4 μm. The lyophilised preparation is particularly suitable for administration using an inhaler, for example commercially available mesh nebulizers comprising, without being limiting, PARI eFlow rapid, PARI LC STAR, PARI Velox and PARI Velox Junior (PARI GmbH, Starnberg, Germany), Philips Respironics I-neb and Philips InnoSpire Go (Koninklijke Philips N. V., Eindhoven, Netherlands), VENTA-NEB-ir, OPTI-NEB, M-neb dose+ mesh nebulizer MN-300/8 or 300/9, M-Neb Flow+ and M-neb mesh nebulizer MN-300/X (NEBU-TEC, Eisenfeld, Germany), Hcmed Deepro HCM-86C and HCM860 (HCmed Innovations Co., Ltd, Taipei, Taiwan), OMRON MicroAir U22 and U100 (OMRON, Kyoto, Japan), Aerogen Solo, Aerogen Ultra and AerogenPRO (Aerogen, Galway, Ireland), KTMED NePlus NESMI (KTMED Inc., Seoul, South Korea), Vectura Bayer Breelib (Bayer AG, Leverkusen, Germany), MPV Truma and MicroDrop Smarty (MPV MEDICAL GmbH, Kirchheim, Germany), MOBI MESH (APEX Medical, New Taipei City, Taiwan), B.Well WN-114, TH-134, and TH-135 (B.Well Swiss AG, Widnau, Switzerland), Babybelle Asia BBU01 (Babybelle Asia Ltd., Hongkong), CA-MI Kiwi and others (CA-MI sri, Langhirano, Italy), Diagnosis PRO MESH (Diagnosis S.A., Białystok, Poland), DIGI 02 (DigiO2 International Co., Ltd., New Taipei City, Taiwan), feellife AIR PLUS, AEROCENTRE+, AIR 360+, AIR GARDEN, AIRICU, AIR MASK, AIRGEL BOY, AIR ANGEL, AIRGEL GIRL and AIR PRO 4 (Feellife Health Inc., Shenzhen, China), Hannox MA-02 (Hannox International Corp., Taipei, Taiwan), Health and Life HL 100 and HL100A (HEALTH & LIFE Co., Ltd., New Taipei City, Taiwan), Honsun NB-810B (Honsun Co., Ltd., Nantong, China), K-jump KN-9100 (K-jump Health Co., Ltd., New Taipei City, Taiwan), microlife NEB-800 (Microlife AG, Widnau, Switzerland), OK Biotech Docspray (OK Biotech Co., Ltd., Hsinchu City, Taiwan), Prodigy Mini-Mist (Prodigy Diabetes Care, LLC, Charlotte, USA), Quatek NM211, NE203, NE320 and NE403 (Big Eagle Holding Ltd., Taipei, Taiwan), Simzo NBM-1 and NBM-2 (Simzo Electronic Technology Ltd., Dongguan, China), Mexus BBU01 and BBU02 (Tai Yu International Manufactory Ltd., Dongguan, China), TaiDoc TD-7001 (TaiDoc Technology Co., New Taipei City, Taiwan), Vibralung and HIFLO Miniheart Circulaire II (Westmed Medical Group, Purchase, USA), KEJIAN (Xuzhou Kejian Hi-Tech Co., Ltd., Xuzhou, China), YM-252, P&S-T45 and P&S-360 (TEKCELEO, Valbonne, France), Maxwell YS-31 (Maxwell India, Jaipur, India), Kernmed JLN-MB001 (Kernmed, Durmersheim, Germany), Yuwell M102 (Yuwell, Nanjing, China), Scian NB-812B (Scian, Shanghai, China).

The lyophilised product can be rehydrated in sterile distilled water or any other suitable liquid for inhalation administration.

Alternatively, for intravenous administration the lyophilised product can be rehydrated in sterile distilled water or any other suitable liquid for intravenous administration.

After rehydration for administration in sterile distilled water or another suitable liquid the lyophilised preparation should have the approximate physiological osmolality of the target tissue for the rehydrated combination preparation i.e., blood for intravenous administration or lung tissue for inhalation administration. Thus, it is preferred that the rehydrated formulation is substantially isotonic.

The preferred dosage concentration for either intravenous, oral, or inhalation administration is between 100 to 2000 μmole/ml, and more preferably is between 200 to 800 μmole/ml.

The manufactured medicaments of the invention comprise:
a) A daily dose of 200 μg of Aviptadil split in three inhalations;
b) A daily dose of 200 μg of Aviptadil split in three inhalations, combined with a daily dose of 54 milligram of Alpha Lipoic Acid split in three inhalations;
c) A daily dose of 200 μg of Aviptadil split in three inhalations, combined with a daily dose of 54 milligram of Alpha Lipoic Acid, orally administered as a drink.

### Method of Treatment

Another aspect of the present invention relates to a method of prophylaxis and/or treatment of an infectious disease, an autoimmune disease, a fibrotic disease, an inflammatory disease, a neurodegenerative disease, or a heart and vascular disease as a consequence of a viral infection such as COVID-19 disease comprising administering to a patient in need thereof a pharmaceutical composition comprising a drug combination according to the present invention.

Accordingly, the terms “prophylaxis” or “treatment” includes the administration of the drug combination of the present invention to prevent, inhibit, or arrest the symptoms of an infectious disease, an autoimmune disease, a fibrotic disease, an inflammatory disease, a neurodegenerative disease, or a heart and vascular disease. In some instances, treatment with the drug combination of the present invention will be done in combination with other protective compounds to prevent, inhibit, or arrest the symptoms of an infectious disease, an autoimmune disease, a fibrotic disease, an inflammatory disease, a neurodegenerative disease, or a heart and vascular disease.

The term “active agent” or “therapeutic agent” as used herein refers to an agent that can prevent, inhibit, or arrest the symptoms and/or progression of an infectious, an autoimmune disease, a fibrotic disease, an inflammatory disease, a neurodegenerative disease, or a heart and vascular disease.

The term “therapeutic effect” as used herein, refers to the effective provision of protection effects to prevent, inhibit, or arrest the symptoms and/or progression of an infectious, an autoimmune disease, a fibrotic disease, an inflammatory disease, a neurodegenerative disease, or a heart and vascular disease as a consequence of COVID-19 disease.

The term “a therapeutically effective amount” as used herein means a sufficient amount of one or more of the drug candidates of the invention to produce a therapeutic effect, as defined above, in a subject or patient in need of treatment.

The terms “subject” or “patient” are used herein mean any mammal, including but not limited to human beings, including a human patient or subject to which the compositions of the invention can be administered.

The drug combination of the present invention can be used for the prophylaxis and/or treatment of an infectious disease, an autoimmune disease, a fibrotic disease, an inflammatory disease, a neurodegenerative disease, or a heart and vascular disease as a consequence of COVID-19 disease in combination administration with another therapeutic compound. As used herein the term “combination administration” of a compound, therapeutic agent or known drug with the drug combination of the present invention means administration of the drug and the one or more compounds at such time that both the known drug and drug combination will have a therapeutic effect. In some cases, this therapeutic effect will be synergistic. Such concomitant administration can involve concurrent (i.e., at the same time), prior, or subsequent administration of the drug with respect to the administration of the drug combination of the present invention. A person of ordinary skill in the art would have no difficulty determining the appropriate timing, sequence and dosages of administration for particular drugs and drug combination of the present invention.

### Drugs of a Combination

The following drugs were tested for the activity as a therapeutic agent for the prophylaxis and/or treatment of an infectious disease, an autoimmune disease, a fibrotic disease, an inflammatory disease, a neurodegenerative disease, or a heart and vascular disease as a consequence of COVID-19 disease:

A peptide having the amino acid sequence: His-Ser-Asp-Ala-Val-Phe-Thr-Asp-Asn-Tyr-Thr-Arg-Leu-Arg-Lys-Gln-Met-Ala-Val-Lys-Lys-Tyr-Leu-Asn-Ser-Ile-Leu-Asn (or Aviptadil).
and the molecule having the IUPAC name:
(R)-5-(1,2-Dithiolan-3-yl)pentanoic acid, (or Alpha Lipoic Acid).

Furthermore, the present invention relates to the use of the above-mentioned drugs as pharmaceutically active agents in medicine, i.e., as medicament.

In order to reach the deep lung where the most post-COVID-19 syndrome symptoms originate from, it is necessary to formulate the medications accordingly. For this we have used a nebulizer which is capable to deliver the respective drugs into the deep lung and into alveoli, and we have formulated the Alpha Lipoic Acid in a solubilized format which is capable of targeting the respective cells in the lungs reaching extremely good bioavailability there.

The terms “a,” “an,” and “the” are used herein to refer to one or to more than one (i.e., to at least one, or to one or more) of the grammatical object of the article.

The term “or” should be understood to mean either one, both, or any combination thereof of the alternatives.

The term “and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Reference throughout this specification to “one embodiment,” “an embodiment,” “a particular embodiment,” “a related embodiment,” “a certain embodiment,” “an additional embodiment,” “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The invention will be further described in the following examples, which do not limit the scope of the invention described in the claims.

### EXAMPLES

The drug compounds as listed above were applied to patients in need after suffering from COVID-19 disease.

### Example 1

We manufactured an inhaled version of Aviptadil in 0.9% NaCl, which can reach the deep lung with high efficacy and so regenerate the disturbed lung physiology. In addition, we manufactured a solubilized version of the Alpha Lipoic Acid that can reach the cells in the deep lung and regenerate the disturbed mitochondria in order to regenerate the ATP production in diseased cells.

### Example 2

A male patient, born in the year 1963 survived a severe COVID-19 disease. Post-COVID-19 symptoms comprised chronic cough, chronic fatigue and a manifested thrombosis in the right leg. Treatment with inhaled combination of Aviptadil (200 μg) and Alpha Lipoic Acid (54 mg) was initiated one week after discharge from hospital due to the fact that the symptoms did not resolve. The patient inhaled a combination of 100 μg of Aviptadil, and 150 mg of Alpha Lipoic Acid split into two inhalation sessions per day; one in the morning and one in the evening. The therapy continued for 3 consecutive months. At the end of the treatment period, the patient was completely free of cough, had no fatigue, and thrombosis resolved.

### Example 3

A male patient, born in the year 1949 survived a severe COVID-19 disease. He was in the intensive care unit with need for intermitted oxygenation between 2-4 l O₂ per minute. Prior to discharge from hospital, the patient showed a typical COVID-19 thorax picture in CT scan, CRP value of 28.9 mg/dl, severe cough and chest pain. At room air the patient had a blood oxygenation value of pO₂ 90 mmHg. As three days after discharge the symptoms did not resolve, the patient inhaled a combination of 200 micrograms of Aviptadil, and 54 milligrams of Alpha Lipoic Acid split into two inhalation sessions per day; one in the morning and one in the evening. The therapy continued for 3 consecutive weeks. At the end of the treatment period, the patient was completely free of cough, free of chest pain, and the blood oxygenation value increased to pO₂ 97 mmHg. After a control visit with the supervising medical doctor, the patient is healthy without any further symptoms.

### Example 4

A male patient, born in the year 1964 survived a severe COVID-19 disease. He was on intensive care unit with need for intermitted oxygenation between 4-7 l O₂ per minute. Prior to discharge from hospital, the patient showed a typical COVID-19 thorax picture in CT scan, and had severe difficulties to breath. At room air the patient had a blood oxygenation value of pO₂ 92 mmHg and needed intermitted oxygenation. Aviptadil inhalation therapy was started in the hospital at day 5 of the stay administering 200 micrograms of Aviptadil split into three inhalation sessions per day. Seven days later, the patient was off external oxygen supply and at room air the patient had a blood oxygenation value of pO₂ 96 mmHg. After discharge from hospital, the post COVID-19 therapy with inhaled Aviptadil continued for 3 consecutive weeks. At the end of the treatment period, the patient breathed normally, and the blood oxygenation value increased to pO₂ 97 mmHg. After a control visit with the supervising medical doctor, the patient is considered healthy.

### Example 5

A male patient, born in the year 1966 survived a severe COVID-19 disease. He was hospitalized, and after being discharged from the hospital he was unable to continue his professional work due to extreme fatigue, cough, and a blood oxygenation value at room air of pO₂ 93 mmHg. Aviptadil inhalation therapy was started at day 7 after hospital discharge, administering 200 micrograms of Aviptadil split into three inhalation sessions per day. The post COVID-19 therapy with inhaled Aviptadil continued for 4 consecutive weeks. At the end of the treatment period, the patient breathed normally, returned to his professional work, started physical exercises like jogging, which was completely impossible after hospital discharge. The blood oxygenation value increased at room air to pO₂ 98 mmHg. After control visit at the supervising medical doctor, the patient is considered healthy without any further post-COVID symptoms.

### Example 6

For comparative purposes, a female patient, born in the year 1981 survived a severe COVID-19 disease. Post-COVID-19 symptoms comprised severe chronic fatigue. The patient refused combination therapy of 100 micrograms of Aviptadil, and 150 milligrams of Alpha Lipoic Acid, and still continues to suffer from severe chronic fatigue.

### Example 7

The presence of VPAC receptors, i.e. vasoactive intestinal peptide receptors, has been shown on airway epithelial cells, on macrophages surrounding capillaries and in the subintima of pulmonary arteries and veins. Stimulation of VPAC receptors leads to the activation of the CAMP and cGMP pathways. Immunocytochemistry and immunohistochemistry for Aviptadil and VPAC receptors: Human pulmonary arterial smooth muscle cells (PASMCs) are fixed, rinsed, and incubated for unspecific protein blocking. Primary Ab's are mouse anti-VPAC-1 immunoglobulins or mouse anti-VPAC-2, both diluted 1:100 in Ab diluents in a moist chamber for 50 min at 37° C. Secondary Ab's are FITC-labeled goat anti-mouse immunoglobulins. Slides are rinsed in PBS and mounted in antifade medium containing DAPI (0.1 μg/ml). Tissue rehydrated paraffin sections are stained for Aviptadil and VPAC receptors. Following antigen retrieval in 5 mM EDTA/PBS (pH 8) for 60 min at 95° C. and unspecific protein blocking in 5% BSA, 0.3% Tween-20, and 0.3% Triton-X 100, the sections are either incubated with rabbit anti-VIP Ab's diluted 1:750 in commercially available Ab diluent for 17 h, or with mouse anti-VPAC-1 immunoglobulins diluted 1:200 in Ab diluent, or with mouse anti-VPAC-2 immunoglobulins diluted 1:100 for 16 h at 4° C. Binding sites were visualized with biotinylated secondary Abs.

Ligand-binding capacity in cultured PASMCs was performed with Aviptadil on VPAC receptors. Ligand binding of 123I-Aviptadil is a function of concentration to membranes of PASMCs cultured from healthy controls and chronically ill patients with lung diseases. Competitive receptor-binding studies with ¹²³I-VIP confirmed the presence of two specific binding sites on primary cell cultures of PASMCs, both in control cells and in patient cells. In control cells, Kd was 25.68±3.11 nM for VPAC1 and 38.55±3.95 nM for VPAC2, respectively, corresponding to a Bₘₐₓ of 0.57±0.05 pM/10⁶ and 0.58±0.04 pM/10⁶ cells, respectively. In contrast, the affinity of Aviptadil binding to its receptors on PASMCs from patients with lung diseases was much higher, with a Kd of 1.70±0.15 for VPAC1 and 0.36±0.13 nM for VPAC2, respectively, and a Bₘₐₓ of 0.89=0.07 pM/10⁶ cells and 0.08±0.01 pM/10⁶ cells.

What is claimed is:

1. Aviptadil, a functional derivative or a precursor thereof, for use in the treatment of a post-viral infection syndrome.

2. A vector encoding Aviptadil, a functional derivative or a precursor thereof, for use of claim 1, for use in the treatment of a post-viral infection syndrome.

3. A biological cell comprising the vector for use of claim 2, for use in the treatment of a post-viral infection syndrome.

4. A composition comprising Aviptadil, the functional derivative or precursor for use of claim 1, the vector for use of claim 2 or the biological cell for use of claim 3, and alpha-lipoic acid, a functional derivative or a precursor thereof, for use in the treatment of a post-viral infection syndrome.

5. A pharmaceutical product comprising Aviptadil, functional derivative or precursor for use of claim 1, the vector for use of claim 2 or the biological cell for use of claim 3, or the composition for use of claim 4, and a pharmaceutically acceptable carrier, excipient and/or diluents for use in the treatment of a post-viral infection syndrome.

6. Aviptadil, the functional derivative or precursor for use of claim 1, the vector for use of claim 2, the biological cell for use of claim 3, the composition for use of claim 4 or the pharmaceutical product for use of claim 5, wherein the treatment of post-viral infection syndrome is the treatment and/or prevention of at least one disease or disorder selected from the group consisting of an autoimmune disease, a fibrotic disease, an inflammatory disease, a neurodegenerative disease, an infectious disease, a lung disease and a heart and vascular disease.

7. Aviptadil, the functional derivative or precursor for use of claim 1 or 6, the vector for use of claim 2 or 6, the biological cell for use of claim 3, the composition for use of claim 4 or 6 or the pharmaceutical product for use of claim 5 or 6, wherein the post-viral infection syndrome is post-COVID-19 syndrome.

8. Aviptadil, the functional derivative or precursor for use of claim 7, the vector for use of claim 7, the biological cell for use of claim 7, the composition for use of claim 7 or the pharmaceutical product for use of claim 7, for use in treatment of at least one symptom of post-COVID-19 syndrome selected from the group consisting of fatigue, somnolence, headaches, dizziness, cerebrovascular disease, seizures, neuropathy and encelopathy.

9. Aviptadil, the functional derivative or precursor for use of any of the previous claims, the vector for use of any of the previous claims, the biological cell for use of any of the previous claims, the composition for use of any of the previous claims or the pharmaceutical product for use of any of the previous claims, for use in oral, intravenous, inhalation and/or intranasal treatment.

10. Aviptadil, the functional derivative or precursor for use of claim 10, the vector for use of claim 10, the biological cell for use of claim 10, the composition for use of claim 10 or the pharmaceutical product for use of claim 10, for use in intranasal treatment.

11. Aviptadil, the functional derivative or precursor for use of claim 11, the vector for use of claim 11, the biological cell for use of claim 11, the composition for use of claim 11 or the pharmaceutical product for use of claim 11, wherein the intranasal administration is direct nose-to-brain administration.

12. A method of treating sarcoidosis in a subject in need thereof, comprising administering an effective amount of Aviptadil to the subject via inhalation or via intravenous administration.

13. The method of claim 12, wherein Aviptadil is administered via inhalation.

14. The method of claim 12, wherein Aviptadil is intravenously administered.
